(19) United States
(12) Patent Application Publication
(10) Pub. No.: US 2004/0039203 A1
(43) Pub. Date: Feb. 26, 2004

(54) Process for the preparation of 1,5-disubstituted-3-amino-1,2,4,-triazoles and substituted aminoguanidines as intermediate compounds

(76) Inventors: Sandor Bokotey, Eztergom (HU); Eva Csikos, Budapest (HU); Csaba Gonczi, Budapest (HU); Felix Hajdu, Budapest (HU); Istvan Hermecz, Budapest (HU); Gergely Heja, Szentendre (HU); Benjamin Pdanyi, Dunakeszi (HU); Andrea Santane Csutor, Budapest (HU); Tiborne Szomor, Budapest (HU); Gyorgyne Szvoboda, Dunakeszi (HU)

Correspondence Address: SANOFI-SYNTHELABO INC., 9 GREAT VALLEY PARKWAY, P.O. BOX 3026, MALVERN, PA, 19355, US

(21) Appl. No.: 10/415,042

(22) PCT Filed: Oct. 25, 2001
(86) PCT No.: PCT/HU01/00103

(30) Foreign Application Priority Data
Oct. 26, 2000 (HU) P0004154

(51) Int. Cl. C07D 241/14

(52) U.S. Cl. 544/386

(57) ABSTRACT

New process for the preparation of 1,5-disubstituted-3-amino-1,2,4-triazoles of the general formula (I), wherein the meaning of R is C₁₋₅alkyl group; C₃₋₁₃cycloalkyl-C₀₋₄alkyl group, optionally substituted by one or more C₁₋₃alkyl group; phenyl-C₀₋₂alkyl-, (CH₂)ₙ-morpholino-, piperidino-, pyrrolidino- or piperazino-group, optionally substituted by one or more halogen atom, C₁₋₃alkyl group, C₁₋₃alkoxy group, n is 1-5, R¹, R², R³, and R⁴ stand independently for hydrogen, halogen, C₁₋₆alkyl group, C₁₋₃alkoxy group or trifuluoromethyl group, with the proviso that of the substituents R¹, R², R³, and R⁴ at least one stands for hydrogen.

The invention relates to a process for the preparation of 1,5-disubstituted-3 -amino-1,2,4-triazoles of the general formula (I),

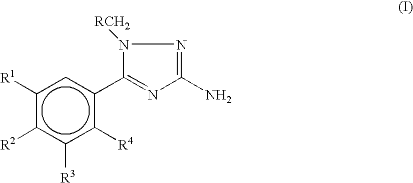

wherein the meaning of R is

C₁₋₅alkyl group; C₃₋₁₃cycloalkyl-C₀₋₄alkyl group, optionally substituted by one or more C₁₋₃alkyl group; phenyl-C₀₋₂alkyl-, (CH₂)ₙ-morpholino-, piperidino-, pyrrolidino- or piperazino-group, optionally substituted by one or more halogen atom, C₁₋₃alkyl group, C₁₋₃alkoxy group,

n is 1-5,

R¹, R², R³, and R⁴ independently stand for hydrogen, halogen, C₁₋₆alkyl group, C₁₋₃alkoxy group or trifluoromethyl group, with the proviso that at least one of the substituents R¹, R², R³, and R⁴ stands for hydrogen.

1,5-disubstituted-3-amino-1,2,4-triazoles of the general formula (I) are intermediates to the therapeutically useful compounds of the general formula (VI),

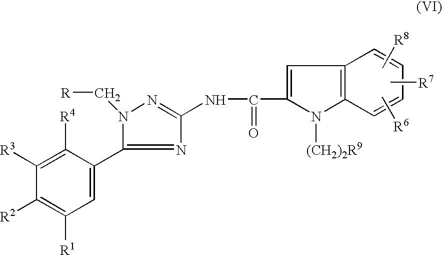

described in patent application WO 98/51686.

For the synthesis of 3-amino-1,2,4-triazoles the route starting from amino-guanidine is known for a long time (K. T. Potts. Chem. Rev. 61, (1961)): an aminoguanidine salt is treated with a suitable acid at high temperature, or the isolated acylaminoguanidine is treated with alkali and/or subjected to thermal ring closure. Except for ring closures in acetic acid, yields are low to medium.

The case is similar when the aminoguanidine is prepared by Schotten-Baumann acylation, where hydrolysis takes place as a competing reaction. The isolated acylaminoguanidine derivative is then cyclized thermally, at 180° C. in DMSO or with NaOEt (J. Med. Chem. 41. 2985-93 (1998)).

Reactions of aminoguanidine with esters leading to 3-amino-1,2,4-triazoles are also known (J. Med. 41, 2985 (1998); and Chem Rev. 61, 87 (1961)).

Synthesis of 1-substituted-3-amino-triazoles cannot be solved economically by preparation and subsequent substitution of the amino-triazoles, since reactivities of the nitrogens in positions 1 and 2 towards electrophiles are almost the same, therefore derivatives substituted in position 1 and in position 2 are equally formed, approximately in the same ratio. Separation of the isomers which have very similar physico-chemical properties is not easy and usually can only be solved by chromatography, which in industrial scale is expensive and complicated.

There is only one example to find in the literature where a substituted aminoguanidine is cyclized to 1,2,4-triazole. The substituted aminoguanidine is synthesized step-by step starting from hydrazide and cyanamide, the ring closure is carried out by thermolysis in pyridine hydrochloride. The synthesis, however, does not utilize some reasonable possibilities given in the aminoguanidine structure [Ger. Offen. 1,808,677 (C.A. 72, 90561m 1970)].

We have found, to our surprise, that the 1,5-disubstituted-3-amino-1,2,4-triazoles of the general formula (I)

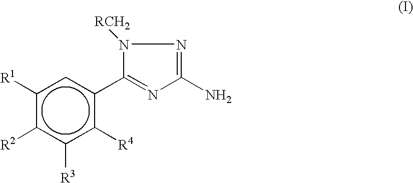

can be prepared in good yield via a regioselective synthesis, by the reaction of the aminoguanidine derivative of the general formula (IV)

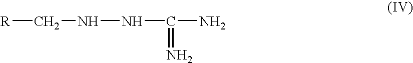

with the acid derivative of the general formula (V),

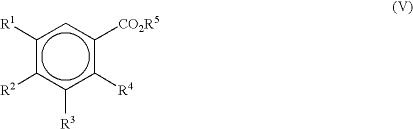

in the presence of sodium- or potassium-alcoholate.

This means that of the four nitrogens of the aminoguanidine, the acylation took place exclusively on the one in desired position. By-products were not obtained even by working up the mother-liquor. A further surprise was that the aminoguanidine, which is sensitive to bases (Houben-Weil VIII, 193.), furnished the product in good yield, this means that decomposition of the aminoguanidine was avoided.

The subject of our invention, in agreement with the above, is a process for the preparation of 1,5-disubstituted-3-amino-1,2,4-triazoles of the general formula (I),

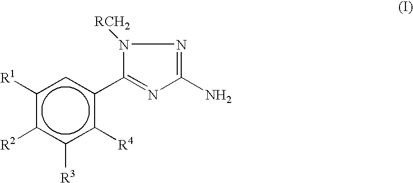

wherein the meaning of R is

C₁₋₅alkyl group; C₃₋₁₃cycloalkyl-C₀₋₄alkyl group, optionally substituted by one or more C₁₋₃alkyl group; phenyl-C₀₋₂alkyl-, (CH₂)ₙ-morpholino-, piperidino-, pyrrolidino- or piperazino-group, optionally substituted by one or more halogen atom, C₁₋₃alkyl group, C₁₋₃alkoxy group,

n is 1-5,

R¹, R², R³, and R⁴ stand independently for hydrogen, halogen, C₁₋₆alkyl group, C₁₋₃alkoxy group or trifluoromethyl group, with the proviso that of the substituents R¹, R², R³, and R⁴ at least one stands for hydrogen, which comprises reacting an aldehyde of the general formula (II)

RCHO (II)

wherein the meaning of R is the same as defined above- with an aminoguanidine of the general formula (III)

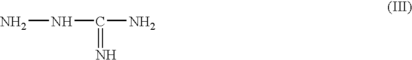

or its salt, and treating the resulting aminoguanidine derivative of the general formula (IV)

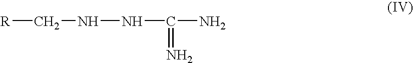

wherein the meaning of R is the same as defined above- with the acid derivative of the general formula (V)

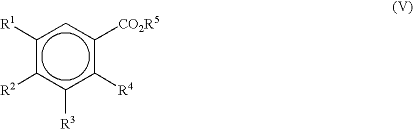

wherein the meanings of R¹, R², R³, and R⁴ are the same as defined above and R⁵ stands for hydrogen atom or for C₁₋₅alkyl group- in the presence of an alkali alcoholate. The aldehydes of formula II are commercially available or they can be prepared by methods known from the literature.

In the process according to the invention for alkali alcoholate preferably sodium- or potassium alcoholate is used.

For compound of the general formula (II) preferably 1-cyclohexylacetaldehyde, and for the acid derivative of the general formula (V) preferably methyl 2,5-dimethoxy-4-methylbenzoate are applied.

Our process is demonstrated by the following examples:

### EXAMPLE 1

51.74g of cyclohexylethylaminoguanidine HCl salt and 49.4g of ethyl 2,5-dimethoxy-4-methylbenzoate are dissolved in 150 ml of methanol, to the solution 28 g of sodium methylate in methanol are added and the reaction mixture is refluxed for 5 hours. After addition of diluted alkali the mixture is refluxed for an additional hour and cooled. The resulting crystals are filtered off, washed and dried, to obtain 55.1 g (67%) of 1-(2-cyclohexylethyl)-5 -(2,5-dimethoxy-4-methylphenyl)-3-amino- 1,2,4-triazole.

Mp.: 136-137° C. Purity by HPLC 99.5%.

Preparation of the Starting cyclohexylethylaaminoguanidine HCl:

11.05 g (0.1 mol) of aminoguanidine hydrochloride are dissolved in 150 ml of 96% ethanol and 12.62 g (0.1 mol) of 1-cyclohexylacetaldehyde are added. The resulting Sclliff-base is hydrogenated under atmospheric pressure, at room temperature, using palladium on charcoal catalyst. At the end the catalyst is filtered off, the filtrate is evaporated, the residue is crystallized from water, to obtain 17.2 g (78%) of product.

Mp.: 132-134° C.

### EXAMPLES 2-43

Applying the procedure as described in Example 1 and using the appropriate starting materials the following products can be prepared (R⁴—H), see Table 1.

TABLE 1

| Example | | | | | Mp. ° C. |
|---|---|---|---|---|---|
| No | R¹ | R² | R³ | RCH₂— | HCl |
| 2 | 2-OCH₃ | 4-CH₃ | 6-OCH₃ | 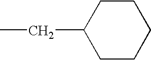 | 135 |
| 3 | 2-OCH₃ | 4-CH₃ | 6-OCH₃ | —CH₂—C₆H₅ | 215 |
| 4 | 2-OCH₃ | 4-CH₃ | 6-OCH₃ | —(CH₂)₄—CH₃ | 143 |
| 5 | 2-OCH₃ | 4-CH₃ | 6-OCH₃ | 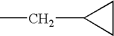 | 236 |
| 6 | 2-OCH₃ | 4-CH₃ | 6-OCH₃ | —CH₂CH₂—C₆H₅ | 200 |
| 7 | 2-OCH₃ | 4-CH₃ | 6-OCH₃ | —(CH₂)₂—CH(CH₃₎₂ | 172 |
| 8 | 2-OCH₃ | 4-CH₃ | 6-OCH₃ | 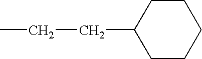 | 187 |
| 9 | 2-OCH₃ | 4-CH₃ | 6-OCH₃ | 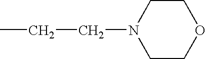 | 160 |
| 10 | 2-OCH₃ | 4-CH₃ | 6-OCH₃ | 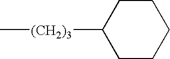 | 190 |
| 11 | 2-OCH₃ | 4-CH₃ | 6-OCH₃ | —(CH₂)₃—CH₃ | 212 |
| 12 | 2-OCH₃ | 4-CH₃ | 6-OCH₃ | 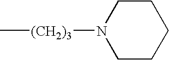 | 198 |
| 13 | 2-OCH₃ | 4-CH₃ | 6-OCH₃ | —CH₂—CH(C₂H₅)₂ | 132 |
| 14 | 2-OCH₃ | 4-CH₃ | 6-OCH₃ | 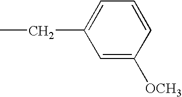 | 197 |
| 15 | 2-OCH₃ | 4-CH₃ | 6-OCH₃ | 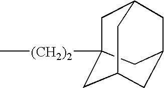 | 217 |
| 16 | 2-OCH₃ | 4-CH₃ | 6-OCH₃ | 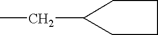 | 208 |
| 17 | 2-OCH₃ | 4-CH₃ | 6-OCH₃ | 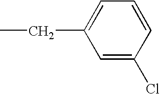 | 136 |
| 18 | 2-OCH₃ | 4-CH₃ | 6-OCH₃ | 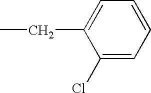 | 204 |
| 19 | 2-OCH₃ | 4-CH₃ | 6-OCH₃ | 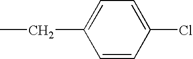 | 202 |
| 20 | 2-OCH₃ | 4-Cl | 5-OCH₃ | 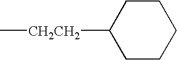 | 196 |
| 21 | 2-OCH₃ | 4-Cl | 5-OCH₃ | 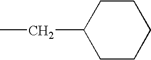 | 148 |
| 22 | 2-OCH₃ | 4-CH₃ | 5-OCH₃ | 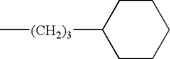 | 192 |
| 23 | 2-OCH₃ | 4-CH₃ | 5-OCH₃ | 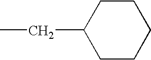 | 188 |
| 24 | 2-OCH₃ | 4-CH₃ | 5-OCH₃ | 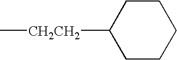 | 166 |
| 25 | 2-OCH₃ | 4-OCH₃ | 6-OCH₃ | 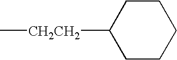 | 189 |
| 26 | 2-OCH₃ | 4-OCH₃ | 6-OCH₃ | 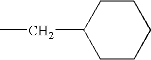 | 180 |
| 27 | 2-OCH₃ | 4-CH₃ | 6-CH₃ | 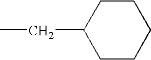 | 168 |
| 28 | 2-OCH₃ | 4-CH₃ | 6-CH₃ | 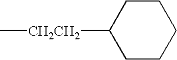 | 188 |
| 29 | 2-OCH₃ | 4-CH₃ | 5-CH₃ | 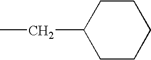 | 200 |
| 30 | 2-OCH₃ | 4-CH₃ | 5-CH₃ | 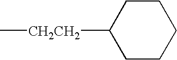 | 206 |
| 31 | 2-OCH₃ | 4-CH₃ | 5-OCH₃ | 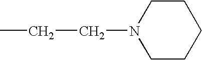 | 218 |
| 32 | 2-OCH₃ | 4-Cl | 5-OCH₃ | 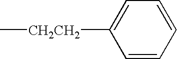 | 127 |
| 33 | 2-OCH₃ | 3-Cl | 6-OCH₃ | 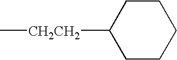 | 159 |
| 34 | 2-OCH₃ | 3-CH₃ | 6-OCH₃ | 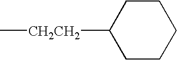 | 168 |

1) Process for the preparation of 1,5-disubstituted-3-amino-1,2,4-triazoles of the general formula (I), 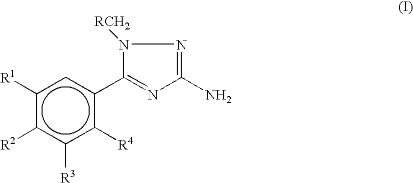
wherein the meaning of R is
C₁₋₅alkyl group; C₃₋₁₃cycloalkyl-C₀₋₄alkyl group, optionally substituted by one or more C₁₋₃alkyl group; phenyl-C₀₋₂alkyl-, (CH₂)ₙ-morpholino-, piperidino-, pyrrolidino- or piperazino-group, optionally substituted by one or more halogen atom, C₁₋₃alkyl group, C₁₋₃alkoxy group,
n is 1-5,
R¹, R², R³, and R⁴ stand independently for hydrogen, halogen, C₁₋₆alkyl group, C₁₋₃alkoxy group or trifluoromethyl group, with the proviso that of the substituents R¹, R², R³, and R⁴ at least one stands for hydrogen,
which comprises
reacting an aldehyde of the general formula (II)
RCHO (II)
wherein the meaning of R is defined above- with an aminoguanidine of the general formula (III) 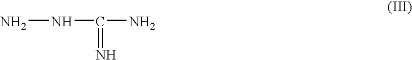
or its salt, and treating the resulting aminoguanidine derivative of the general formula (IV) 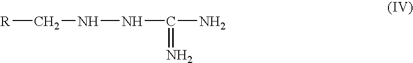
wherein the meaning of R is defined above- with the acid derivative of the general formula (V) 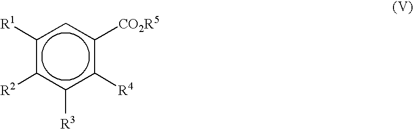
wherein the meanings of R¹, R², R³, and R⁴ are defined above and R⁵ stands for hydrogen atom or for C₁₋₅alkyl group- in the presence of an alkali alcoholate.

2) The process defined in claim 1, which comprises the use of sodium- or potassium alcoholate as alkali alcoholate.

3) The process defined in claim 1, which comprises the use of 1-cyclohexylacetaldehyde for compound of the general formula (II).

4) The process defined in claim 1, which comprises the use of methyl 2,5-dimethoxy-4methylbenzoate for compound of the general formula (V).

5) Compounds of the general formula (IV), 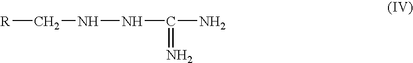
wherein the meaning of R is
C₁₋₅alkyl group; C₃₋₁₃cycloalkyl-C₀₋₄alkyl group, optionally substituted by one or more C₁₋₃alkyl group; phenyl-C₀₋₂alkyl-, (CH₂)ₙ-morpholino-, piperidino-, pyrrolidino- or piperazino-group, optionally substituted by one or more halogen atom, C₁₋₃alkyl group, C₁₋₃alkoxy group.
